(19) United States
(12) Patent Application Publication
(10) Pub. No.: US 2004/0034418 A1
(43) Pub. Date: Feb. 19, 2004

(54) Membrane-reinforced implants

(76) Inventors: Shu-Tung Li, Oakland, NJ (US); Tom Smestad, Palo Alto, CA (US); Julie Pon, Mountain View, CA (US); Robert McNeill, Mill Valley, CA (US)

Correspondence Address: FISH & RICHARDSON PC, 225 FRANKLIN ST, BOSTON, MA, 02110, US

(21) Appl. No.: 10/201,727

(22) Filed: Jul. 23, 2002

(51) Int. Cl. A61F 2/08

(52) U.S. Cl. 623/13.17; 623/13.18; 623/14.12; 623/23.61; 623/23.75; 623/11.11

(57) ABSTRACT

An implant that contains a membrane and a polymeric matrix covered by the membrane. Both the matrix and the membrane are biocompatible and bioresorbable. Also disclosed is a method of preparing such an implant.

### BACKGROUND

Implants are widely used for reconstruction of damaged tissues. Such implants include dental implants, hip and knee implants, plates and pins for broken bones, and other devices. Some of them are successful in reducing the suffering and disabilities associated with tissue damages. However, many of them fail to perform long-term functions, as the implant material deteriorates within a human body. Coating or reinforcement of an implant with an appropriate material can facilitate the joining between the implant and human tissues, and increase the long-term stability and integrity of the implant.

### SUMMARY

The present invention relates to membrane-reinforced implants.

In one aspect, this invention features an implant that contains a membrane and a polymeric matrix covered by the membrane. Both the matrix and the membrane are biocompatible and bioresorbable. Examples of the implant of the invention include a cartilage implant (e.g., a meniscus implant), a ligament implant, a tendon implant, and a bone implant. The matrix of the implant can be a synthetic polymer-based matrix or a biopolymer-based matrix. An example of a biopolymer-based matrix is a collagen-based matrix such as a type I collagen-based matrix. The membrane of the implant can be a synthetic membrane or a biomembrane. Examples of a biomembrane include a pericardium membrane, a small intestine submucosa membrane, and a peritoneum membrane. The surface of the matrix can be covered by the membrane either partially or completely. In particular, for a meniscus implant, the surface of the matrix that faces the femoral condyles can be covered by the membrane.

In another aspect, this invention features a method of preparing an implant described above. The method involves conforming (e.g., trimming) a membrane to a predetermined shape and size, and covering a surface of a polymeric matrix with the membrane. As mentioned above, both the membrane and the matrix are biocompatible and bioresorbable. The membrane can be affixed on the surface of the matrix with various glues. For instance, the membrane can be affixed on the surface of the matrix with a biological glue such as fibrin or a mussel adhesive, or a chemical glue such as cyanoacrylate. The membrane can also be affixed on the surface of the matrix with sutures.

The present invention provides a method of preparing membrane-reinforced implants for reconstruction of damaged tissues in vivo. The details of one or more embodiments of the invention are set forth in the accompanying drawings and description below. Other advantages, features, and objects of the invention will be apparent from the drawings and the detailed description, and from the claims.

### BRIEF DESCRIPTION OF THE DRAWINGS

FIG. 1 is a schematic drawing of a finished medial meniscus matrix implant, wherein the membrane is stabilized with the collagen-based matrix using fibrin glue.

FIG. 2 is a schematic drawing of a finished medial meniscus matrix implant, wherein the membrane is stabilized with the collagen-based matrix using sutures.

### DETAILED DESCRIPTION

The present invention pertains to a membrane-reinforced, polymeric (e.g., biopolymeric) scaffold matrix device. A meniscus implant is described in detail below as an example.

Menisci are crescent shaped fibrocartilages that are anatomically located between the femoral condyles and tibia plateau, providing stability, load distribution, force transmittance and assisting in lubrication of the knee joint. The meniscus has a thickness of about 7 to 8 mm at the periphery and gradually tapers to a thin tip at the inner margin, forming a slightly concave triangle in cross section. The major portion of the meniscal tissue is avascular except the peripheral rim which comprises about 10% to 30% of the total width of the structure and is nourished by the peripheral vasculature. The avascular tissue of the meniscus is composed of fibrochondrocytes surrounded by an abundant extracellular matrix and water (about 70% of the weight of tissue) where the nutrients are provided presumably through physicochemical processes. Collagen accounts for the majority of the matrix material, amounting to about 75% by weight of the dry tissue, whereas the rest is made of non-collagenous proteins and polysaccharides. Approximately 90% of the collagen in meniscus tissue is type I collagen, and the collagen fibers are oriented primarily in the circumferential direction. The anisotropy and lack of homogeneity in the structure are consistent with the complexity of the in vivo biomechanical functions of the menisci.

Injury to the knee, commonly occurring in athletes, frequently results in the tear of meniscus tissue. Repair of the torn tissue in the peripheral vascular rim can be accomplished arthroscopically with sutures or similar technique where the wound usually heals with the return of normal meniscus functions. However, in more severe cases where the injured site is in the avascular region where the repair of the damaged tissue is often inadequate or impossible, partial or total removal of the damaged meniscus tissue is often indicated.

Studies in animals and in humans have shown that removal of the meniscus is a prelude to degenerative knees manifested by the development of degenerative arthritis. The development of degenerative arthritis on meniscectomized knees is consistent with force distribution analysis of the knee which shows that menisci of the knee joint play a significant role in load distribution and transmission. Thus, removal of meniscus tissue results in a redistribution of the load, leading to a greater force concentration of the opposing articular surfaces.

Attempts have been made to replace the resected meniscus tissue with a biological or synthetic material. Autografts, allografts and various synthetic materials have all been tested. Each of these materials has some merit and can partially fulfill the requirements of a meniscus substitute. However, none of these materials has demonstrated long-term efficacy in vivo. While short-term results of allografting appear encouraging, long-term fate of allografts remains unknown. In addition, many disadvantages associated with allografting require further attention.

Most of the synthetic materials used for meniscus replacement are intended to function as a permanent prosthesis. It is known that most polymeric materials are subjected to mechanical fatigue and degradation under continuous cyclic stress and strain applications. Typically in the knee joint where there are several million cycles of loading and unloading of multiple body weights, the ultimate failure of the meniscus substitute can be anticipated. The degradation of the material can result in not only loss of mechanical function, but particle generated can cause adverse tissue reactions. In addition, none of the materials can simulate the mechanical properties of the intact meniscus to function effectively in vivo. Furthermore, the joint may be further traumatized as a result of redistribution of the load due to mismatch of the mechanical properties.

In the prior art, Stone (U.S. Pat. Nos. 5,007,934, 5,116,374 and 5,158,574) and Li, et al. (U.S. Pat. Nos. 5,681,353, 5,735,903 and 6,042,610) used type I collagen to fabricate a meniscus implant device that served as a scaffold to support the meniscus tissue regeneration. The device was successfully tested in humans. Even though the implant can provide patients with potential long-term benefit, the device requires a substantial period of rehabilitation during the healing of the implant. Therefore, patients receiving the implant are inconvenienced for several months. The long period of rehabilitation also introduces the risk of tear of the implant during the wound healing and new tissue regeneration. In order to shorten the rehab time, minimizing the potential damage to the implant and improving the quality of life sooner, the material characteristics of the meniscus implant have to be improved. Since the prior art meniscus was prepared from reconstitution of collagen fibers, it lacked certain mechanical properties to withstand repetitive shear stresses, particularly at the inner margin of the implant which is thin and weak (FIG. 1). In order to prevent the shear-related damage to the implant during the initial healing, a composite implant can be used to provide the necessary mechanical properties to serve the function of a meniscus regeneration scaffold without sacrificing other essential requirements.

A meniscus implant of this invention is used to support the meniscus tissue regeneration in the human knee joint. The device has a dimension similar to the size of a human meniscus and can be trimmed by the surgeon to fit the size of the meniscus defect during the surgery. The device has the necessary physical and physico-chemical characteristics for supporting meniscus tissue regeneration.

The suitable biopolymeric materials for the present invention include proteins and polysaccharides. Proteins useful for the present invention include collagen-based materials, elastin-based materials, and the like. The polysaccharides useful for the present invention include cellulose, alginic acid, chitin and chitin derivatives, and the like. In one example, the implant device is made of collagen-based material. Type I collagen fibers can be used for this application due to their biocompatibility and availability. Type I collagen can be obtained from any type I collagen-rich tissues of human and animal. Genetically-engineered type I collagen can also be used for this purpose.

The method for fabricating a scaffold has been described in the prior art (U.S. Pat. Nos. 5,007,934 and 5,735,903) and is incorporated herein as if set out in full. In particular, an acid dispersion of type I collagen fibers is prepared and the fibers are coacervated with an alkaline solution such as an ammonium hydroxide or a sodium hydroxide solution. The coacervated fibers are partially dehydrated and molded into a predetermined size and shape of defined density. The mold used for the present invention has a dimension similar to a human medial or lateral meniscus. Typically, for a medial meniscus implant, the mold has a dimension of approximately 80% of an averaged human meniscus. This size is similar to a subtotal resection during partial meniscectomy procedure, leaving a 2 to 3 mm vascular peripheral meniscal rim intact for the attachment of the implant device and for the infiltration of host cells and nutrient into the scaffold matrix. For a lateral meniscus, the dimension of the mold is slightly modified to accommodate the anatomical difference between menisci. The molded fibers are then lyophilized. The procedure for lyophilizing a porous collagen-based matrix is well known in the art. For a meniscus implant of the present invention, the matrix is lyophilized at −20° C. under a vacuum of less than 400 milli-torr for about 48 hours, followed by drying under vacuum for about 12 to 24 hours at about 20° C. The lyophilized matrix is then cross-linked using a crosslinking agent commonly employed by medical implant manufacturers such as glutaraldehyde, formaldehyde or any other bifunctional agents that can react with amino, carboxyl, hydroxyl and guanidino groups of proteins and polysaccharides. Formaldehyde vapor is frequently used for cross-linking the porous collagen-based materials due to its volatility and therefore can be used for cross-linking the meniscus implant.

A biocompatible and bioresorbable membrane is then attached to the fabricated matrix using a biocompatible glue to stabilize the membrane with the matrix. Useful glues for this application include fibrin glue, cyanoacrylate and bio-adhesive derived from mussels or barnacles from the ocean. Alternatively, the membrane may be stabilized with the matrix using sutures. Any resorbable or non-resorbable sutures may be used for this purpose. Biological membranes useful for this application include pericardium tissues from animals or humans, small intestine submucosa from animals, peritoneum, or the like. The membranes may be used to cover a portion or the entire surface of the implant in contact with the articular surface of the femoral condyles to prevent the potential shear-induced damage to the implant in vivo. The membrane can be perforated to increase the permeability of the membrane to cells. Perforated holes have a diameter greater than 50 μm such that cells and their associated processes can infiltrate through the membrane without mechanical interference.

The meniscus implant of the present invention can be used as a meniscus regeneration scaffold, for implantation into a defect (e.g., a segmental defect) of a meniscus in a subject. A segmental meniscus defect typically encompasses a tear or lesion (including radial tear, horizontal tear, bucket handle tears, complex tears) in less than the entire meniscus, resulting in partial resection of the meniscus. Upon implantation into a segmental defect of a meniscus, the composite formed by the partial meniscus and the scaffold device has an in vivo outer surface contour substantially the same as a whole natural meniscus without a segmental defect, and establishes a biocompatible and bioresorbable scaffold adapted for ingrowth of meniscal fibrochondrocytes.

Accordingly, the present invention provides a method for regenerating a meniscus tissue in vivo. The method involves fabricating a meniscus repair implant device composed of a composite of biocompatible and bioresorbable matrix as described above, and a biocompatible and resorbable membrane sheet, and then implanting the device into a segmental defect in the meniscus. The implanted device establishes a biocompatible and bioresorbable scaffold adapted for ingrowth of meniscal fibrochondrocytes. The scaffold, in combination with the ingrown chondrocytes, supports natural meniscus load forces.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

### EXAMPLE 1

### Preparation of Biological Membrane

Bovine pericardium was obtained from a USDA approved abattoir. The tissue was cleaned by scraping away the adhered fatty tissue and other extraneous materials. The pericardium was rinsed with 300 ml of water for 2 hours at room temperature, followed by soaking in 300 ml of 1% Triton X-100 for 24 hours at 4° C. The pericardium was then defatted in 300 ml of isopropanal for 2 hours and again in 300 ml isopropanal overnight at room temperature. The isopropanol-rinsed pericardium was then washed twice in water and stored at 4° C. until use.

### EXAMPLE 2

### Preparation of Membrane-Reinforced Meniscus Implant—Method I

A 0.7% of type I collagen fiber dispersion in 0.07 M lactic acid solution was first prepared. Aliquot of the dispersion was weighed into a flask and the pH adjusted to about 4.8 to 5.0 to coacervate the fibers. The coacervated fibers were partially dehydrated and inserted into a mold. A piece of pericardium tissue from Example 1 was cut to size and placed on the surface (facing the femoral condyles in vivo) of partially dehydrated matrix, and the pericardium membrane was integrated with the matrix by applying a weight over the top of the membrane. The molded fibers were then freeze-dried for 48 hours at −20° C. and a vacuum of about 100 millitorr, followed by drying at 20° C. and a vacuum of about 100 milli-torr for 18 hours. The freeze-dried matrix was cross-linked with formaldehyde vapor generated from 2% formaldehyde solution for about 30 hours to stabilize the matrix. The matrix was rinsed and dried in air.

Dexon suture (Ethicon, Sommerville, N.J.) was used to suture the membrane with the matrix using interrupting techniques to further stabilize the pericardium membrane with the matrix implant.

### EXAMPLE 3

### Preparation of Membrane-reinforced Meniscus Implant—Method II

A 0.7% of type I collagen dispersion in 0.07 M lactic acid solution was first prepared. Aliquot of the dispersion was weighed into a flask and the pH adjusted to about 4.8 to 5.0 to coacervate the fibers. The coacervated fibers were partially dehydrated and inserted into a mold.

The molded fibers were then freeze-dried for 48 hours at −20° C. and a vacuum of about 100 millitorr, followed by drying at 20° C. and a vacuum of about 100 milli-torr for 18 hours. The freeze-dried matrix was cross-linked with formaldehyde vapor generated from 2% formaldehyde solution for 30 hours to stabilize the matrix. The matrix was rinsed and dried in air.

A piece of pericardium tissue was cut to size and commercial fibrin glue (CryoLife, Marietta, Ga.) was applied to the surface of the membrane and the matrix, and the membrane was stabilized with the matrix via light pressure over the membrane.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. An implant comprising:
a membrane, and
a polymeric matrix covered by the membrane,
wherein both the matrix and the membrane are biocompatible and bioresorbable.

2. The implant of claim 1, wherein the implant is a cartilage implant, a ligament implant, a tendon implant, or a bone implant.

3. The implant of claim 2, wherein the matrix is a biopolymer-based matrix.

4. The implant of claim 3, wherein the membrane is a biomembrane.

5. The implant of claim 4, wherein the membrane is a pericardium membrane, a small intestine submucosa membrane, or a peritoneum membrane.

6. The implant of claim 3, wherein the matrix is a collagen-based matrix.

7. The implant of claim 6, wherein the matrix is a type I collagen-based matrix.

8. The implant of claim 6, wherein the membrane is a biomembrane.

9. The implant of claim 8, wherein the membrane is a pericardium membrane, a small intestine submucosa membrane, or a peritoneum membrane.

10. The implant of claim 2, wherein the implant is a meniscus implant.

11. The implant of claim 10, wherein the matrix is a biopolymer-based matrix.

12. The implant of claim 11, wherein the membrane is a biomembrane.

13. The implant of claim 12, wherein the membrane is a pericardium membrane, a small intestine submucosa membrane, or a peritoneum membrane.

14. The implant of claim 11, wherein the matrix is a collagen-based matrix.

15. The implant of claim 14, wherein the matrix is a type I collagen-based matrix.

16. The implant of claim 14, wherein the membrane is a biomembrane.

17. The implant of claim 16, wherein the membrane is a pericardium membrane, a small intestine submucosa membrane, or a peritoneum membrane.

18. The implant of claim 10, wherein the surface of the matrix that faces the femoral condyles is covered by the membrane.

19. A method of preparing an implant, the method comprising:
conforming a membrane to a predetermined shape and size, and
covering a surface of a polymeric matrix with the membrane,
wherein both the membrane and the matrix are biocompatible and bioresorbable.

20. The method of claim 19, wherein the membrane is affixed on the surface of the matrix with a biological glue.

21. The method of claim 20, wherein the biological glue is fibrin.

22. The method of claim 20, wherein the biological glue is a mussel adhesive.

23. The method of claim 19, wherein membrane is affixed on the surface of the matrix with a suture.

24. The method of claim 19, wherein membrane is affixed on the surface of the matrix with a chemical glue.

25. The method of claim 24, wherein the chemical glue is cyanoacrylate.

26. The method of claim 19, wherein the implant is a cartilage implant, a ligament implant, a tendon implant, or a bone implant.

27. The method of claim 26, wherein the implant is a meniscus implant.

28. The method of claim 27, wherein the surface of the matrix that faces the femoral condyles is covered by the membrane.

29. The method of claim 19, wherein the matrix is a biopolymer-based matrix.

30. The method of claim 29, wherein the matrix is a collagen-based matrix.

31. The method of claim 30, wherein the matrix is a type I collagen-based matrix.

32. The method of claim 19, wherein the membrane is a biomembrane.

33. The method of claim 32, wherein the membrane is a pericardium membrane, a small intestine submucosa membrane, or a peritoneum membrane.
